(19) United States
(12) Patent Application Publication
(10) Pub. No.: US 2026/0061002 A1
(43) Pub. Date: Mar. 5, 2026

(54) METHOD AND SYSTEM FOR TREATMENT OF BONE OR JOINT

(71) Applicant: REDDRESS LTD., Pardes Hana (IL)

(72) Inventor: Alon KUSHNIR, Givat Ada (IL)

(73) Assignee: REDDRESS LTD., Pardes Hana (IL)

(21) Appl. No.: 19/106,285

(22) PCT Filed: Aug. 31, 2023
(86) PCT No.: PCT/IL2023/050932

§ 371 Date: Feb. 25, 2025

(30) Foreign Application Priority Data
Aug. 31, 2022 (IL) 296102

(51) Int. Cl. A61K 35/14; A61K 45/06

(52) U.S. Cl. CPC A61K 35/14; A61K 45/06

(57) ABSTRACT

Whole blood is mixed with one or more coagulating agents to yield a coagulating blood that is then introduced into a site of bone or join injury or disease and permitted to coagulate in said site. The purpose is to facilitates healing or amelioration of a disease, disorder or injury at said site. For example, healing and regeneration of damaged bones, damaged joints, cartilage lesions, etc.

### TECHNOLOGICAL FIELD

The present disclosure is generally in the field of orthopedics and concerns a formulation and method for inducing regenerative processes of bones or joints.

### BACKGROUND ART

While the skeleton has a regenerative capacity, there are occasional injuries such as bone damage and joint wear, that do not heal and necessitate a medical intervention procedure. Such are, for example a variety of complex bone damages that are caused by disease or injury, where the body natural repair mechanisms are unable to heal the damage, or damages to the joint as a result of mechanical wear or prolonged infections, e.g., as a result of osteoarthritis or rheumatoid arthritis.

In the case of bone damages, a particular procedure often used is the grafting of a bone graft to replace the bone that was lost due to a disease, fracture, etc. The underlying therapeutic principles include osteoconduction (for guiding growth of the natural bone), osteoinduction (for encouraging undifferentiated cells to become active osteoblasts), and osteogenesis (where the graft material includes living bone cells that contribute to bone remodeling). While osteoconduction and osteoinduction can involve a variety of different grafts, osteogenesis only occurs with autograft tissue and allograft cellular bone matrices. Other bone grafting materials include demineralized bone matrix (DMB), synthetic materials, minerals (such as hydroxyapatite), composite of organic polysaccharides (such as chitin, chitosan and alginate) and others.

A joint injection (intra-articular injection) of a therapeutic agent, e.g., an anti-inflammatory agent, is a common treatment of inflammatory joint conditions, such as in rheumatoid arthritis, psoriatic arthritis, gout, tendinitis, bursitis, Carpal Tunnel Syndrome and in osteoarthritis. Injections into the joint also include lubricants, such as hyaluronic acid that, because of its high viscosity, is sometimes used to replace bursa fluids.

A wound dressing comprising a blood coagulant has been suggested for the treatment of chronic wounds. See WO2010/086848, WO2019/058373, WO2019/150355, WO2019/058375 and WO2021/124317. Use of a coagulating blood for treatment of various damages tissues has been disclosed in WO2022/168099, WO2022/180627 and WO2023/037373.

### GENERAL DESCRIPTION

The present disclosure provides a method for treating a disease, disorder or injury of bone or joint.

The term “condition” is used herein to collectively denote a disease, disorder or injury.

The term “site” is used herein to denote the site where the condition is manifested, for example an infected joint, a severed or fractured bone site, a bone or joint site that underwent a surgical procedure, etc.

The term “blood” is used herein (interchangeably with “whole blood”) to denote whole blood, typically venous blood withdrawn from the treated subject.

The term “coagulating blood” is used herein to denote whole blood that is still in a flowable state and is in the process of coagulation. A coagulating blood is typically whole blood mixed with one or more of a coagulating agent or an anti-anti-coagulant (namely an agent that counteracts or inhibits the action of an anti-coagulant with which whole blood is mixed when withdrawn from a subject to avoid its coagulation).

According to this disclosure, whole blood is mixed with one or more coagulating agents and the coagulating blood is then introduced into said site and permitted to coagulate in said site. Provided by one aspect of this disclosure is a method for treating a bone or joint disease or injury in a subject in which a mixture of whole blood and one or more coagulating agents is introduced into the site of disease or injury to permit the blood to coagulate in said site. Provided by another aspect is a system comprising elements, including one or more devices and/or materials for carrying out the method. Also provided by further aspects of this disclosure is an assembly and a kit of parts comprising one or more of the elements of said system or elements that are needed for carrying out said method.

The method of this disclosure facilitates healing or amelioration of said condition. For example, healing and regeneration of damaged bones, damaged joints, cartilage lesions, etc.

According to this disclosure, a mixture of whole blood and one or more coagulating agents is introduced to said site and permitted to coagulate in situ. Typically, the whole blood is mixed, prior to its introduction to said site, with one or more coagulating agents that causes the blood to coagulate, and prior to complete coagulation, while the blood is still in a flowable state (namely in a liquid or partially coagulated state) it is introduced to said site.

The blood may, by some embodiments, mixed with other agents such as bone substitutes or bone regeneration inducers, ant-inflammatory agent, disease modifiers, etc.

### EMBODIMENTS

Some embodiments of this disclosure will now be described in the following numbered paragraphs and referred to in subsequent paragraphs by reference to the numbers (in a format similar to that of claims). These embodiments intend to add onto (and not limit) the disclosure herein:
1. A method for treating a bone or joint disease or injury in a subject, comprising:
introducing a mixture of whole blood and one or more coagulating agents into the site of disease or injury and permitting the blood to coagulate in said site.
2. The method of embodiment 1, comprising
withdrawing blood from the subject,
mixing the withdrawn blood with one or more coagulating agents to thereby form said mixture, and
introducing the mixture into said site.
3. The method of embodiment 2, wherein
the withdrawn blood is mixed with an anti-coagulant and said one or more coagulating agents comprises an anti-anti-coagulant, being an agent that counters the anti-coagulating effect of the anti-coagulant.
4. The method of embodiment 3, wherein said one or more coagulating agents comprises kaolin.
5. The method of any one of embodiments 1-4, wherein said one or more coagulating agents comprises a calcium salt of an organic acid (e.g., calcium gluconate).
6. The method of any one of claims 1-5, wherein said introducing is by injection.
7. The method of any one of embodiments 1-6, for treating a bone injury, comprising
adding a bone affecting agent into said mixture, said agent having the effect of inducing one or more of (i) bone regenerative processes, (ii) amelioration of damage or injury to the bone tissue, and (iii) ameliorating symptoms of disease or damage of the bone, and
introducing said mixture into said site.
8. The method of embodiment 7, wherein said bone-affecting agent is a bone graft material.
9. The method of embodiment 8, wherein the bone graft material is selected from material with one or more of the bone-regeneration effects that include osteoconduction, osteoinduction and osteogenesis.
10. The method of embodiment 9, wherein the bone graft material is selected from the group consisting of bone tissue, demineralized bone matrix, synthetic materials, minerals and composite organic polysaccharides.
11. The method of any one of embodiments 1-6, for treating an inflammatory joint disease, cartilage injury or cartilage lesions.
12. The method of embodiment 11, comprising adding a joint-affecting therapeutic agent to said mixture.
13. The method of embodiment 12, comprising
adding a joint affecting agent into said mixture, said agent having the effect of inducing one or more of (i) joint regenerative processes, (ii) amelioration of damage or injury to the joint tissue, and (iii) ameliorating symptoms of disease or damage of the joint, and
introducing said mixture into the joint.
14. The method of any one of embodiments 11-13, for the treatment of an inflammatory joint disease.
15. The method of embodiment 14, comprising injecting said mixture into the joint.
16. A system for treating a joint or bone injury or disease, comprising
one or more devices or combination of devices for withdrawing and collecting whole blood,
a vessel configured for mixing the whole blood with one or more coagulating agents, and
one or more devices for introducing the blood mixture into the site of disease or injury.
17. The system of embodiment 16, comprising one or more coagulating agents.
18. The system of embodiment 17, wherein
the one or more device for collecting the whole blood comprises an anti-coagulant, and wherein
said one or more coagulating agents comprises an anti-anti-coagulant, being an agent that counters the anti-coagulating effect of the anti-coagulant.
19. The system of embodiment 18, wherein said anti-anti-coagulant comprises kaolin.
20. The system of any one of embodiments 16-19, wherein said one or more coagulating agents comprises a calcium salt of an organic acid (e.g., calcium gluconate).
21. The system of any one of embodiments 16-20, wherein said one or more coagulating agents is comprised in said vessel.
22. The system of any one of embodiments 16-21, comprising a syringe for injecting blood from said vessel into a site of manifestation of said injury or disease.
23. The system of any one of embodiments 16-22, for treating a bone disease or injury, comprising
a bone affecting agent intended for mixing with whole blood, said agent having the effect of inducing one or more of (i) bone regenerative processes, (ii) amelioration of damage or injury to the bone tissue, and (iii) ameliorating symptoms of disease or damage of the bone
24. The system of embodiment 23, wherein said bone-affecting agent in a bone graft material.
25. The system of embodiment 24, wherein the bone graft material is selected from material with one or more of the bone-regeneration effects that include osteoconduction, osteoinduction and osteogenesis.
26. The system of embodiment 25, wherein the bone graft material is selected from the group consisting of bone tissue, demineralized bone matrix, synthetic materials, minerals and composite organic polysaccharides.
27. The system of any one of embodiments 16-22, for treating an inflammatory joint disease, cartilage injury or cartilage lesions.
28. The system of embodiment 27, comprising a joint-affecting therapeutic agent for adding to the whole blood.
29. The system of embodiment 28, wherein
said joint affecting agent has the effect of inducing one or more of (i) joint regenerative processes, (ii) amelioration of damage or injury to the joint tissue, and (iii) ameliorating symptoms of disease or damage of the joint.
30. The system of any one of embodiments 27-29, for use in the treatment of an inflammatory joint disease.
31. An assembly or a kit of parts comprising one or more of the elements define in any one of embodiments 16-30.

### DETAILED DESCRIPTION

The method for treating a joint or bone condition in a subject, according to this disclosure, comprises introducing a mixture of whole blood and one or more coagulating agents into the site of disease or injury and permitting the blood to coagulate in said site.

The term “blood mixture” will be used to denote the mixture of blood with the one or more coagulating agents, and optionally with other agents such as agents having a therapeutic effect by their own right including anti-inflammatory drugs, antibiotics or other antimicrobial drugs, drugs with a disease-modifying effect and others.

The term “mixing vessel” will be used, herein to denote a vessel in which the blood is mixed with other agents.

The blood mixture is introduced to the treatment site such that the coagulation process is initiated ex vivo, namely outside the body of the patient and is completed in vivo, namely at the treatment site. The blood mixture is typically introduced to the treatment site in a liquid or semi-jelly pre-clotted state and the coagulation process is completed in said site such that the formulation is fully coagulated in situ at said site.

Typically, the rate of coagulation may be controlled, for example by the nature or concentration of the one or more coagulating agents.

The one or more coagulating agents may comprise a single agent or a combination of agents that induce blood coagulation. The anti-anticoagulant is an agent that counteracts the anti-coagulant that is mixed with blood after its withdrawal to inhibit its coagulation. When blood (usually venous blood) is withdrawn into a blood collection tube, e.g., a vacutainer, in the absence of an anticoagulant it will quickly coagulate. To avoid its coagulation the withdrawn blood is, typically, mixed with an anticoagulant, such as sodium citrate, ACD-A (Anticoagulant Citrate Dextrose, Solution A), heparin, oxalate, EGTA or EDTA, and consequently remains flowable. In order to induce a coagulation reaction before introduction of the blood to said site, an anti-anticoagulant, namely an agent that counteracts the coagulation inhibiting effect of the coagulating such as calcium salts of organic acids, e.g., calcium gluconate, may be admixed into the blood.

In addition, or in the alternative, the one or more coagulating agents may comprise an agent that accelerates the blood-clotting cascade, increase firmness of the formed blood coagulant, etc. such as kaolin, glass powder, celite powder, ellagic acid, silica powder. As can be appreciated, the teaching of this disclosure is not limited to any specific coagulating agent or a combination of coagulating agents.

By one embodiment, said one or more coagulating agents comprises kaolin and calcium gluconate.

The blood may be mixed with the one or more coagulating agents in a dedicated mixing vessel which may be configured for that purpose. The mixing vessel may, at times, be the same vessel in which the withdrawn blood was collected. The mixing vessel may also be the device used for transferring the blood into said site, such as the barrel of a syringe.

The mixing vessel may a priori comprise within it, the one or more coagulating agents. Alternatively, the one or more coagulating agents may be introduced prior, jointly or after the introduction of the blood into this vessel. By still another alternative, at least one of the one or more coagulating agents may be comprised with said vessel and at least one other of the one or more coagulating agents may be introduced prior or after the introduction of the blood into this vessel.

By an embodiment of this disclosure, the blood is an autologous blood withdrawn from the subject. The blood may be used immediately after it is withdrawn or may be stored for a period of time, under appropriate conditions, until use.

The blood mixture may be introduced to said site by injection. Alternatively, by other embodiments, or example in the case of a site of an open bone fracture, the blood mixture may be pasted into said site.

By one embodiment of treating a bone condition in accordance with this disclosure, the blood mixture is combined with a bone-affecting agent, which may be an agent having the effect of inducing one or more of (i) bone regenerative processes, (ii) amelioration of damage or injury to the bone tissue, and (iii) ameliorating symptoms of disease or damage of the bone. An example of a bone-affecting agent is a bone graft material, e.g., a material having one or more of the bone-regeneration effects that include osteoconduction, osteoinduction and osteogenesis. Specific examples of such bone graft material are bone tissue (from a donor or cadaver), demineralized bone matrix, synthetic materials, minerals and composite organic polysaccharides. By some embodiment, the blood mixture with the bone-affecting agent may also comprise anti-inflammatory agents, anti-microbial agents (e.g., antibiotics) and others.

By another embodiment, the mixture is used for treating a joint condition including, for example, an inflammatory joint disease, cartilage injury or cartilage lesions. By some embodiments a joint-affecting therapeutic agent may be added to said mixture. Such a joint-affecting agent may be an agent having the effect of inducing one or more of (i) joint regenerative processes, (ii) amelioration of damage or injury to the joint tissue, and (iii) ameliorating symptoms of disease or damage of the joint. By some embodiment, the blood mixture with the joint-affecting therapeutic agent may also comprise anti-inflammatory agents, anti-microbial agents (e.g., antibiotics) and others.

Provided by another aspect of this disclosure is a system for carrying out the method. The system of this disclosure comprises one or more devices or combination of devices for withdrawing and collecting the blood; a mixing vessel; and comprises one or more devices for introducing the blood mixture into said site.

The one or more devices or combination of devices for withdrawing and collecting the blood may be standard blood withdrawal and collection devices routinely used in laboratory practice, such as an evacuated tube system consisting of a venipuncture needle, a tube holder that may be coupled directly to the needle or through a linking tubing, and an evacuated tube. However, this disclosure is not limited to any specific device or a combination of devices for blood withdrawal and collection.

The mixing vessel may be the same or different than the blood collection device or the device for introducing the blood mixture to said site. By some embodiments, blood collection vessel, typically a blood collection tube, may also be used as the mixing vessel. By other embodiments, the mixing vessel is a dedicated vessel configured specifically for that purpose. By yet another embodiment the mixing vessels is the same as that used for transfer of the blood mixture to said site and thus serves this dual purpose.

The system may also comprise said one or more coagulating agents. Said one or more coagulating agents may be comprised within said mixing vessel, may be comprised in an independent container to be added to mixing vessel prior, jointly or after the introduction of the blood into this vessel.

The one or more devices for introducing the blood mixture into said site may be a syringe or any other device or a combination of devices useful for that purpose.

The system may also comprise, contingent with the intended use, a bone-affecting agent or a joint-affecting agent to be combined with said mixture, a therapeutic drug, etc.

Also provided by additional aspects of this disclosure is an assembly or a kit of parts comprising one or more of the elements for use in the above method or comprised by said system.

1-15. (canceled)

16. A method for treating a bone or joint disease or injury in a subject, comprising:
introducing a mixture of whole blood and one or more coagulating agents into the site of disease or injury and permitting the blood to coagulate in said site.

17. The method of claim 16, comprising
withdrawing blood from the subject,
mixing the withdrawn blood with one or more coagulating agents to thereby form said mixture, and
introducing the mixture into said site.

18. The method of claim 7, wherein
the withdrawn blood is mixed with an anti-coagulant and said one or more coagulating agents comprises an anti-anti-coagulant, being an agent that counters the anti-coagulating effect of the anti-coagulant.

19. The method of claim 16, for treating a bone injury, comprising
adding a bone affecting agent into said mixture, said agent having the effect of inducing one or more of (i) bone regenerative processes, (ii) amelioration of damage or injury to the bone tissue, and (iii) ameliorating symptoms of disease or damage of the bone, and
introducing said mixture into said site.

20. The method of claim 16, for treating an inflammatory joint disease, cartilage injury or cartilage lesions.

21. The method of claim 20, comprising
adding a joint affecting agent into said mixture, said agent having the effect of inducing one or more of (i) joint regenerative processes, (ii) amelioration of damage or injury to the joint tissue, and (iii) ameliorating symptoms of disease or damage of the joint, and
introducing said mixture into the joint.

22. The method of claim 20, for the treatment of an inflammatory joint disease.
